(19) United States
(12) Patent Application Publication
(10) Pub. No.: US 2016/0102336 A1
(43) Pub. Date: Apr. 14, 2016

(54) FLUORESCENT PROBE FOR DETECTING ACTIVITY OF CALPAIN

(71) Applicant: THE UNIVERSITY OF TOKYO, Bunkyo-ku, Tokyo (JP)

(72) Inventors: Tetsuo NAGANO, Tokyo (JP); Kenjiro HANAOKA, Tokyo (JP); Yu KUSHIDA, Tokyo (JP)

(73) Assignee: THE UNIVERSITY OF TOKYO, Tokyo (JP)

(21) Appl. No.: 14/772,576

(22) PCT Filed: Mar. 4, 2014
(86) PCT No.: PCT/JP2014/055482

§ 371 Date: Dec. 8, 2015

(30) Foreign Application Priority Data
Mar. 4, 2013 (JP) 2013-042382

(51) Int. Cl. C12Q 1/37

(52) U.S. Cl. CPC C12Q 1/37; G01N 2333/96466

(57) ABSTRACT

[Problem]

To provide a novel fluorescent probe for detecting the activity of calpain.

[Solution]

A compound represented by general formula (I) or a salt thereof.

### TECHNICAL FIELD

The present invention relates to a red fluorescent probe capable of detecting the activity of calpain.

### BACKGROUND ART

Calpain, which is a type of cysteine protease, is enzyme-activated with dependence on Ca²⁺ concentration and is an important modulator molecule for regulating various cell functions via limited decomposition of the substrate. The intracellular activity thereof is strictly controlled by a protein called calpastatin. Calpain is ubiquitously present in cells in vivo, and known examples include calpain-1 (μ-calpain) and calpain-2 (m-calpain), which differ in Ca²⁺ concentration required for the enzyme activation. These are present as 80-kDa+30-kDa heterodimers. Calpain is deeply involved, in particular, in the regulation of cell migration and cell death, and there have been an increasing number of reports in recent years suggesting a relationship between loss of control of calpain activity and transformation of neurodegenerative diseases or cancer malignancy. Also, calpain is involved in multiple sclerosis, muscular dystrophy, Alzheimer's disease, and other nerve and muscle disorders for which there are few effective drugs, and there is growing interest in calpain as a potential drug target. Visualization of calpain activity is important for the understanding of disease mechanisms and drug discovery research.

It is important to detect changes in calpain activity in living cells in association with various imparted stimulations and/or gene knockdown in order to elucidate the involvement of calpain in disorders. Currently, a blue fluorescent probe is mainly used for detecting calpain activity in living cells. However, these blue fluorescent probes create various problems when they are used. More specifically, it has been reported that (1) they are difficult to be used in a system that requires high tissue transparency since the self-fluorescence is high due to bio-molecules included in biomedical tissue, individual animals, and other measurement samples; (2) although calpain activity and Ca²⁺ concentration in cells can be preferably observed simultaneously, a combination use of a blue fluorescent probe and Fura-2, which is a Ca²⁺ probe, is impossible; and (3) a blue fluorescent probe undergoes discoloration upon exposure to UV irradiation when NP-EGTA is de-caged, NP-EGTA being a caged compound that releases Ca²⁺ with dependence on the radiation of light.

Thus, conventional fluorescent probes for detecting calpain activity have various problems, and there have yet to be any reports of a fluorescent probe for detecting calpain activity in which these problems have been solved.

### SUMMARY OF THE INVENTION

### Problems to be Solved by the Invention

An object of the present invention is to provide a novel fluorescent probe for detecting calpain activity.

### Means Used to Solve the Above-Mentioned Problems

After thoroughgoing research, the present inventors thought it possible to contribute considerably to the advancement of calpain research by adding a novel red region to the detection of calpain activity to thereby broaden multicolor imaging of calpain and other bio-molecules through the use of Fura-2 and caged compounds, as well as labelling of substrate by green fluorescent proteins (GFP). As a result, the present inventors achieved the present invention having found that prior art problems can be solved in a red fluorescent probe having as a mother nucleus a compound resulting from substituting with a silicon atom the oxygen atom of pyronin Y (PY), which is the base backbone of rhodamine.

In other words, the present invention provides:

[1] a compound represented by general formula (I) below or a salt thereof.

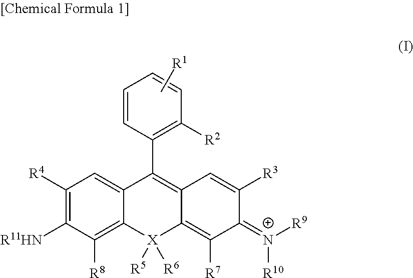

(where:

R¹ is a hydrogen atom or represents 1 to 4 monovalent substituents present on a benzene ring, which are the same or different;

R² is a monovalent substituent;

R³ and R⁴ are, independently, a hydrogen atom or C₁₋₆ alkyl group;

R⁵ and R⁶ are, independently, a C₁₋₆ alkyl group or aryl group;

R⁷ and R⁸ are, independently, a hydrogen atom or C₁₋₆ alkyl group;

R⁹ and R¹⁰ are, independently, a hydrogen atom or C₁₋₆ alkyl group;

R⁹ or R¹⁰ optionally forms, together with R³ or R⁷, a 5-7-member heterocyclyl or heteroaryl containing a nitrogen atom to which R⁹ or R¹⁰ binds, and optionally contains 1-3 additional heteroatoms selected from the group consisting of O, N, and S as ring-constituting members; the heterocyclyl or heteroaryl optionally being substituted by a C₁₋₆ alkyl, C₂₋₆ alkenyl, C₂₋₆ alkynyl, C₆₋₁₀ aralkyl, or C₆₋₁₀ aralkenyl group;

R¹¹ is a monovalent substituent cleaved by contact with calpain; and

X is a silicon, germanium, or tin atom.

[2] The compound or salt thereof according to [1], wherein R¹¹ is a monovalent substituent containing an oligopeptide residue.
[3] The compound or salt thereof according to [2], wherein the monovalent substituent containing an oligopeptide residue is expressed by formulas (1), (2), or (3) below.

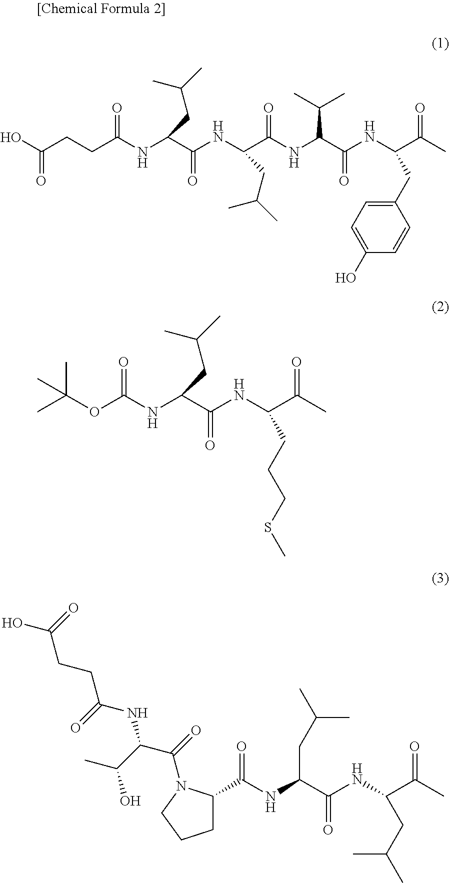

[4] The compound or salt thereof of any of [1] to [3], wherein X is a silicon or germanium atom.
[5] A compound represented by formula (4) below, or salt thereof.

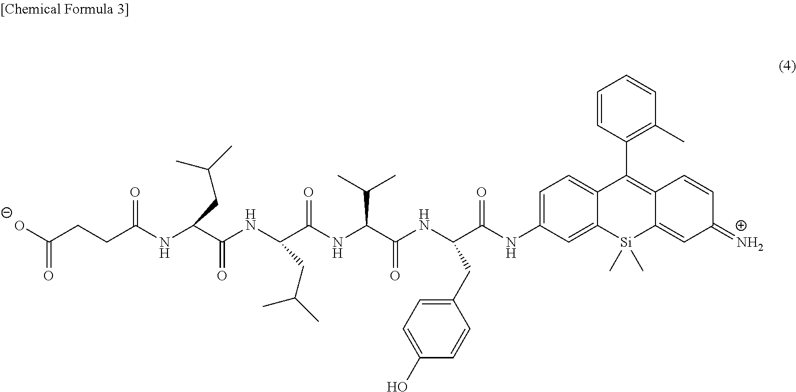 [SMILES: Cc1ccccc1C1=C2C=CC(=[NH2+])C=C2[Si](C)(C)c2cc(NC(=O)[C@H](Cc3ccc(O)cc3)NC(=O)[C@@H](NC(=O)[C@H](CC(C)C)NC(=O)[C@H](CC(C)C)NC(=O)CCC(=O)[O-])C(C)C)ccc21]

[6] A compound represented by formula (5) below, or salt thereof.

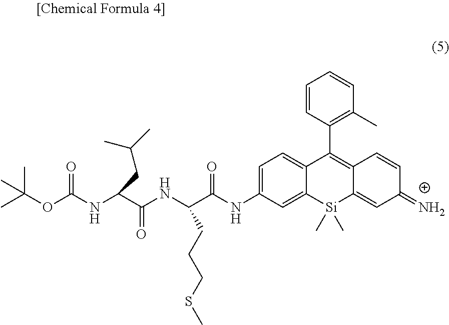 [SMILES: CSCCC[C@H](NC(=O)[C@H](CC(C)C)NC(=O)OC(C)(C)C)C(=O)Nc1ccc2c(c1)[Si](C)(C)C1=CC(=[NH2+])C=CC1=C2c1ccccc1C]

[7] A compound represented by formula (6) below, or salt thereof.

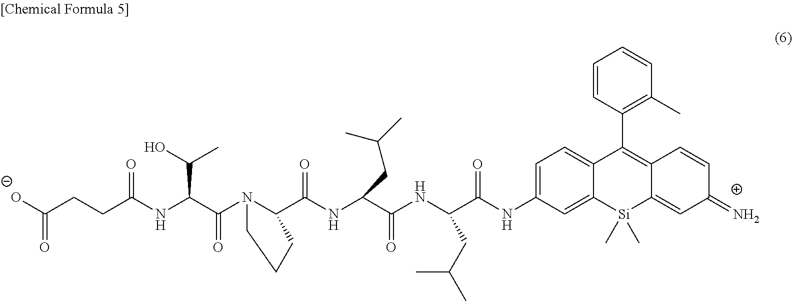 [SMILES: Cc1ccccc1C1=C2C=CC(=N)C=C2[Si](C)(C)c2cc(NC(=O)[C@H](CC(C)C)NC(=O)[C@H](CC(C)C)NC(=O)[C@@H]3CCCN3C(=O)[C@@H](NC(=O)CCC(=O)[O-])C(C)O)ccc21]

[8] A fluorescent probe containing the compound or salt thereof according to any one of claims [1] to [7].
[9] A method for measuring calpain, comprising the following steps of:

(a) bringing the compound or salt thereof according to any one of [1] to [7] and calpain into contact with each other, and

(b) measuring the fluorescence intensity of the compound generated in step (a) after contact with calpain.

### Advantages of the Invention

Using the compound of the present invention makes it possible to provide a fluorescent probe having excellent optical stability and capable of detecting calpain activity in a long-wavelength region. The compound of the present invention also makes it possible to broaden multicolor imaging of calpain and other bio-molecules through the use of Fura-2 and caged compounds, as well as labelling of substrate by green fluorescent proteins (GFP).

### BRIEF DESCRIPTION OF THE DRAWINGS

FIG. 1 Results of evaluation of Suc-LLVY-SiR600 as a fluorescent probe.

FIG. 2 Results of evaluating Boc-LM-SiR600 as a fluorescent probe.

FIG. 3 Images of calpain activity in HeLa cells using Suc-LLVY-SiR600.

FIG. 4 Images of calpain activity in A549 cells using Suc-LLVY-SiR600.

FIG. 5 Costaining by Suc-LLVY-SiR600 and Lyso Tracker.

FIG. 6 Costaining by 2Me SiR600 and Lyso Tracker.

### BEST MODE FOR CARRYING OUT THE INVENTION

In the present invention, unless otherwise noted, “alkyl group” or an alkyl moiety of a substituent (e.g., an alkoxy group or the like) containing an alkyl moiety refers to a C₁₋₆, preferably C₁₋₄, and more preferably C₁₋₃ alkyl group comprising a straight chain, branched chain, ring, or combination thereof. More specifically, examples of the alkyl group include a methyl group, ethyl group, n-propyl group, isopropyl group, cyclopropyl group, n-butyl group, sec-butyl group, isobutyl group, tert-butyl group, cyclopropylmethyl group, n-pentyl group, and n-hexyl group. In the present specification, the term “halogen atom” may be a fluorine atom, chlorine atom, bromine atom, or iodine atom, and is preferably a fluorine atom, chlorine atom, or bromine atom.

One embodiment of the present invention is a compound represented by general formula (I) below, or salt thereof.

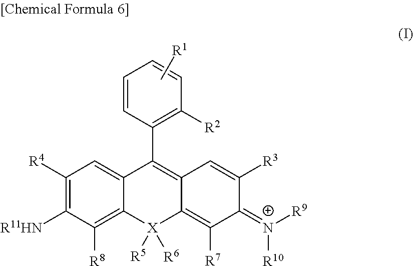

In general formula (I), R¹ is a hydrogen atom or represents one to four monovalent substituent groups present on a benzene ring, which are the same or different. When R¹ represents a monovalent substituent group present on a benzene ring, it is preferred that one or two same or different substituent groups be present on the benzene ring. When R¹ represents one or more monovalent substituent groups, the substituent group can be substituted at any position on the benzene ring. Preferably, all of R¹ represent a hydrogen atom, or a single substituent group is present (R¹ other than the substituent group are hydrogen atoms).

The type of monovalent substituent group represented by R¹ is not particularly limited; preferred examples may be selected from the group consisting of a C₁₋₆ alkyl group, a C₁₋₆ alkenyl group, a C₁₋₆ alkynyl group, a C₁₋₆ alkoxy group, a hydroxy group, a carboxy group, a sulfonyl group, an alkoxycarbonyl group, a halogen atom, and an amino group. These monovalent substituent groups may furthermore have any of one or more substituent groups. For example, one or more halogen atoms, carboxy groups, sulfonyl groups, hydroxy groups, amino groups, alkoxy groups, or the like may be present in the alkyl group represented by R¹, and, for example, the alkyl group represented by R¹ may be an alkyl halide group, a hydroxyalkyl group, a carboxyalkyl group, an aminoalkyl group, or the like. Also, one or two alkyl groups may be present in the amino group represented by R¹, and the amino group represented by R¹ may be a monoalkyl amino group or a dialkyl amino group. Furthermore, in the case that the alkoxy group represented by R¹ has a substituent group, examples thereof include a carboxy-substituted alkoxy group and an alkoxycarbonyl-substituted alkoxy group, and more specific examples include a 4-carboxybutoxy group and a 4-acetoxymethyloxycarbonylbutoxy group.

In general formula (I), R² represents a monovalent substituent group. There are no particular limitations as to the type of monovalent substituent group represented by R²; in similar fashion to R¹, preferred examples may be selected from the group consisting of a C₁₋₆ alkyl group, a C₁₋₆ alkenyl group, a C₁₋₆ alkynyl group, a C₁₋₆ alkoxy group, a hydroxy group, a carboxy group, a sulfonyl group, an alkoxycarbonyl group, a halogen atom, and an amino group.

In general formula (I), R³ and R⁴ independently represent a hydrogen atom, a C₁₋₆ alkyl group, or a halogen atom. When R³ or R⁴ represents an alkyl group, one or more halogen atoms, carboxy groups, sulfonyl groups, hydroxy groups, amino groups, alkoxy groups, or the like may be present in the alkyl group, and, for example, the alkyl group represented by R³ and R⁴ may be an alkyl halide group, a hydroxyalkyl group, a carboxyalkyl group, or the like. R³ and R⁴ are, independently, preferably a hydrogen atom or a halogen atom, and it is more desirable that R³ and R⁴ both be hydrogen atoms, or R³ and R⁴ both be chlorine atoms or fluorine atoms.

In general formula (I), R⁵ and R⁶ independently represent a C₁₋₆ alkyl group or aryl group. R⁵ and R⁶ are, independently, preferably a C₁₋₃ alkyl group, and R⁵ and R⁶ are both more preferably a methyl group. One or more halogen groups, carboxy groups, sulfonyl groups, hydroxy groups, amino groups, alkoxy groups, or the like may be present in the alkyl group represented by R⁵ and R⁶, and the alkyl group represented by R⁵ and R⁶ may be, e.g., an alkyl halide group, a hydroxyalkyl group, a carboxyalkyl group, or the like. When R⁵ or R⁶ represents an aryl group, the aryl group may be a monocyclic aromatic group or condensed aromatic group, and the aryl ring may include one or more ring-structured heteroatoms (e.g., nitrogen atom, sulfur atom, oxygen atom, or the like). A phenyl group is preferred as the aryl group. One or more substituent groups may be present on the aryl ring. One or more substituent groups, e.g., a halogen atom, carboxy group, sulfonyl group, hydroxy group, amino group, alkoxy group, or the like may be present.

In general formula (I), R⁷ and R⁸ are, independently, a hydrogen atom, C₁₋₆ alkyl group, or halogen atom, of which explanations described for R³ and R⁴ are also applicable to R⁷ and R⁸. R⁷ and R⁸ are preferably both hydrogen atoms, both chlorine atoms, or both fluorine atoms.

In general formula (I), R⁹ and R¹⁰ are, independently, a hydrogen atom or a C₁₋₆ alkyl group. R⁹ or R¹⁰ optionally forms, together with R³ or R⁷, a 5-7-member heterocyclyl or heteroaryl containing a nitrogen atom to which R⁹ or R¹⁰ binds, and optionally contains 1-3 additional heteroatoms selected from the group consisting of O, N, and S as ring-constituting members; the heterocyclyl or heteroaryl furthermore optionally being substituted by a C₁₋₆ alkyl, C₂₋₆ alkenyl, C₂₋₆ alkynyl, C₆₋₁₀ aralkyl group (benzyl group, phenethyl group, or the like), or C₆₋₁₀ aralkenyl group. Examples of the heterocyclyl or heteroaryl formed in this manner include and are not limited to pyrrolidine, piperidine, hexamethyleneimine, pyrrole, imidazole, pyrazole, oxazole, and thiazole.

In a preferred aspect of the present invention, both R⁹ and R¹⁰ are hydrogen atoms.

In formula (I), R¹¹ is a monovalent substituent to be cleaved by contact with calpain. A monovalent substituent containing an oligopeptide residue is preferred as the monovalent substituent to be cleaved by contact with calpain.

The monovalent substituent containing an oligopeptide residue is preferably one having the sequence Leu-Leu-Val-Tyr, Thr-Pro-Leu-Leu, Leu-Met, Thr-Pro-Leu-Lys, Thr-Pro-Leu-Phe, Leu-Tyr (directly bonded to a NH group in which the amino acid at the right end of the sequence is bonded to pyronin Y (PY)).

The N terminal of the monovalent substituent containing an oligopeptide residue may be protected, and protecting groups that may be used include a succinyl group, a tert-butoxycarbonyl group, and a benzyloxycarbonyl group, but a substituent group other than these may be used.

In an embodiment of the present invention, the monovalent substituent containing an oligopeptide residue is represented by formula (1), (2), or (3) below.

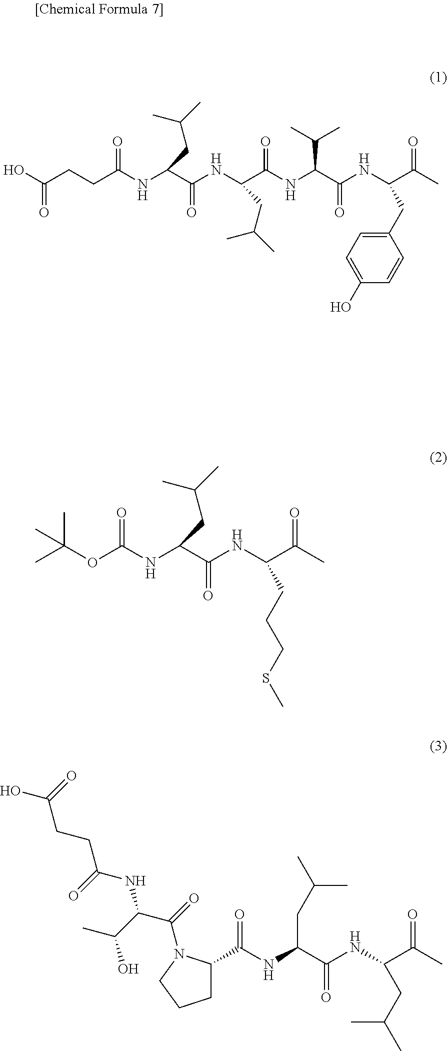

A preferred embodiment of the present invention is the compound represented by the formula (4), (3), or (6) below, or salt thereof.

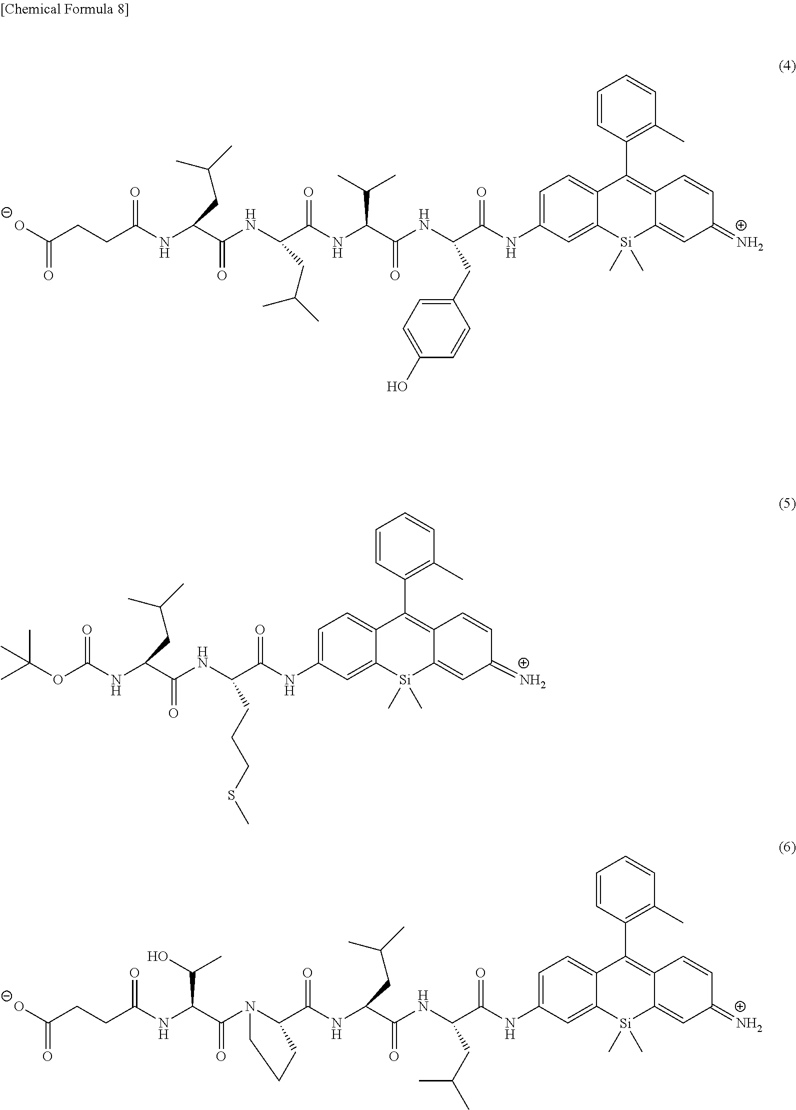

A compound represented by the general formula (I), and formulas (4), (5), or (6) in the present invention may be present as a salt. Examples of a salt include a base-addition salt, an acid-addition salt, and an amino acid salt. Examples of base-addition salts include sodium salt, potassium salt, calcium salt, magnesium salt, and other metal salts, ammonium salt, or triethyl amine salt, piperidine salt, morpholine salt, and other organic amine salts. Examples of acid-addition salts include hydrochlorides, sulfates, nitrates, and other mineral acid salts; and methanesulfonic acid salt, p-toluenesulfonic acid, citrates, oxalates, and other organic acid salts. An example of amino acid salts is glycine salt. As shall be apparent, salts of the compound of the present invention are not limited to the foregoing.

The compound of the present invention represented by general formula (I) may have one or more asymmetric carbons in accordance with the substituent species, and an optical isomer, or diastereoisomer or other stereoisomer may be present. An unadultered stereoisomer, any mixture of stereoisomers, racemates, and the like are also included in the scope of the present invention. The compound of the present invention represented by general formula (I) or salt thereof may also be present as a hydrate or solvate, and all of these substances are included in the scope of the present invention. There are no particular limitations as to the type of solvent that forms the solvate; examples include ethanol, acetone, isopropanol, and other solvents.

The fluorescent probe comprising the compound represented by general formula (I), and formulas (4), (5), and (6) or salt thereof provided by the present invention is capable of generating a compound (corresponding to a compound in which R¹ in general formula (I) is a hydrogen atom) in which the R¹¹ substituent or monovalent substituent containing an oligopeptide residue is cleaved by contact with calpain and the absorption wavelength is shifted, and can be advantageously used as a fluorescent probe for measuring calpain.

Measurement of calpain using the fluorescent probe described above can be carried out in accordance with methods well known to a person skilled in the art, and it is therefore possible to make application as a reagent for diagnosis for animals and humans in addition to use as a reagent for research. For example, the concentration and amount of a substance to be measured in a test tube can be measured using the fluorescent probe described above, or incorporated into living cells or organisms, and then imaged and measured using bio-imaging techniques. Typical examples include a method containing the steps of: (a) bringing calpain into contact with the compound represented by general formula (I) or salt thereof which has a monovalent substituent to be cleaved by contact with calpain; and (b) measuring the fluorescence intensity of the compound generated in step (a) after the contact with calpain.

There are no particular limitations as to the method for using the fluorescent probe of the present invention; examples include: measurement of isolated and purified enzymes, and calpain activity in a cell lysate; measurement of calpain activity in a living cell; and measurement of activity of enzymes acting as cancer biomarkers in biomedical tissue which make use of optical characteristics such as long wavelengths.

### EXAMPLES

The present invention is described in more detail below using examples, but the scope of the present invention is not limited by the examples described below. In the examples, Me refers to a methyl group.

### Examples 1 to 3

Three types of the compound of the present invention were synthesized in accordance with Synthesis Scheme 1 below.

Using 9-o-toluyl-9H-Si-xanthene-3,6-diamine (1) (of which synthesis method is disclosed in PCT/JP2012/53855) as a starting material compound, a compound to which an oligopeptide residue (Leu-Leu-Val-Tyr, Thr-Pro-Leu-Leu, Leu-Met) has been introduced via the steps shown in the following scheme (Suc-LLVY-SiR600:Suc refers to a succinyl group; Suc-TPLL-SiR600 and Boc-LM-SiR600:Boc refers to a tert-butoxycarbonyl group) was synthesized.

### [Chemical Formula 9]

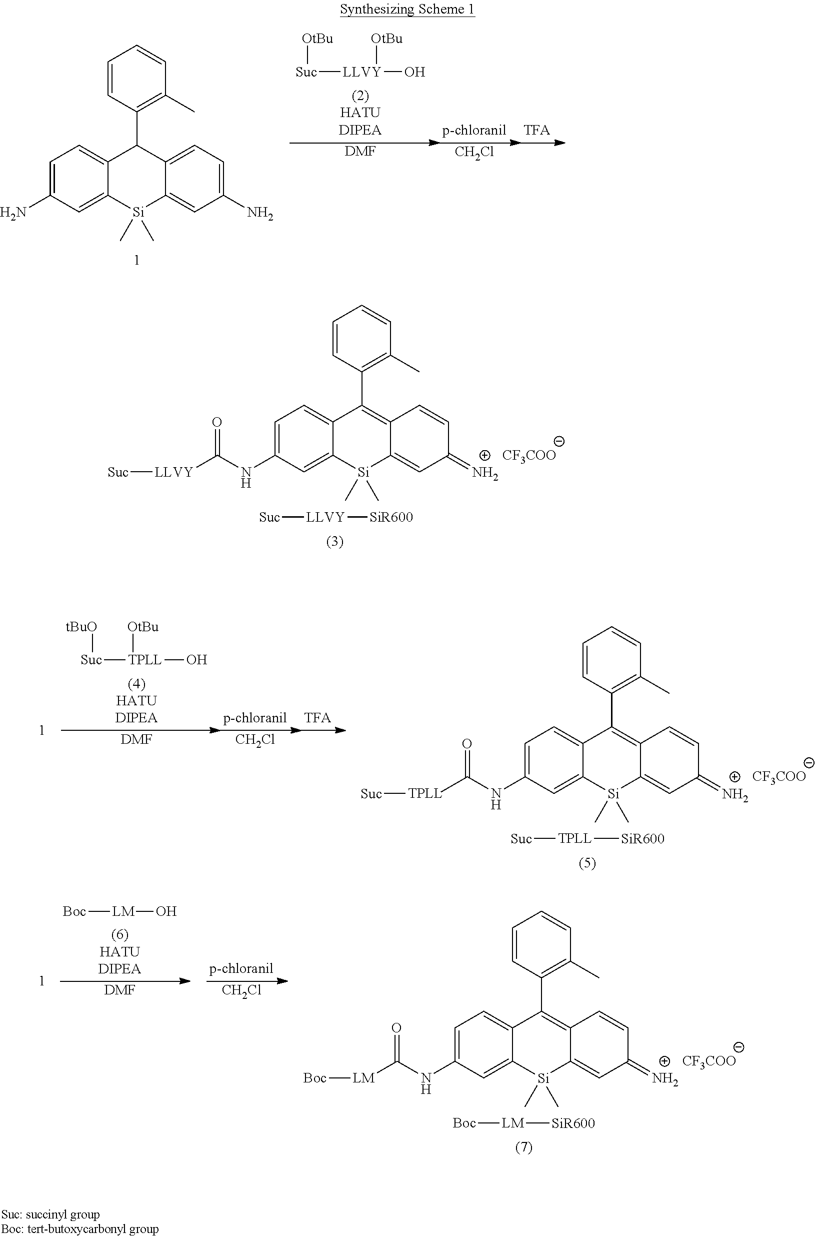

Side-chain-protected peptides (2, 4, 6)

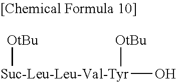

2

HRMS (ESI⁺): m/z Found 741.4450. calculated 741.4415 for [M+Na]⁺ (+3.6 mmu)

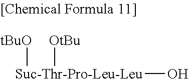

4

HRMS (ESI⁺): m/z Found 677.4097. calculated 677.4102 for [M+Na]⁺ (−0.4 mmu)

Boc-Leu-Met-OH [Chemical Formula 12]
6

HRMS (ESI⁺): m/z Found 385.1773. calculated 385.1811 for [M+Na]⁺ (+3.8 mmu)

Side-chain-protected peptides (2, 4, 6) were synthesized using ordinary Fmoc solid phase synthesis described below using 2-chlorotrityl chloride resin (1.3 mmol/g, 100-200 mesh, 1% DVB).

(a) Peptide-coupling cycle: Fmoc amino acid (5 equivalent of resin) and O-(7-azabenzotriazole-1-yl)-N—N—N′—N′,-tetramethyluronium hexafluorophosphate (HATU: 5 equivalent of resin) were dissolved in DMF, diisopropylethylamine (DIPEA: 10 equivalent of resin) was added and the system was stirred. The resulting solution was added to a resin in which an N-terminal-deprotected peptide has been coupled, and the system was stirred for 40 minutes.
(b) Fmoc deprotection cycle: The Fmoc-protected group was deprotected by adding a 20% (v/v) piperidine/DMF solution to the resin and stirring the system for 12 minutes.
(c) Excision from resin: A solution of trifluoroacetic acid:dichloromethane (2:98) was added to the resin, the system was stirred for 1 minute for 10 cycles to thereby excise the peptide from the resin. The resin was removed by filtration, the filtrate was distilled out under reduced pressure, and an excess amount of cold water was added to the residue to transudate the resulting precipitate; yielding a crude peptide.
(d) Crude peptide refinement: The crude peptide was dissolved in water/acetonitrile, and the solution was refined using reverse-phase HPLC for fractionation; yielding side-chain-protected peptides (2, 4, 6).

### (1) Synthesis of Suc-LLVY-SiR600

### Example 1

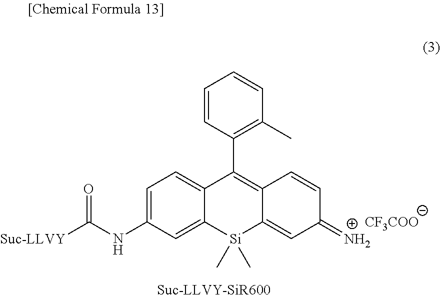

9-o-toluyl-9H-Si-xanthene-3,6-diamin (3.6 mg, 10.5 μmol) was dissolved in DMF (0.5 mL), side-chain-protected peptide 2 (8.3 mg, 11.5 μmol), HATU (8.7 mg, 23.0 μmol), and DIPEA (8 μL, 46.1 μmol) were added, and the system was stirred for 21 hours at room temperature. Water was added to the reaction mixture, the resulting mixture was extracted using dichloromethane and washed using a saline solution. The organic layer was dried using sodium sulfate, and the solvent was then distilled out under reduced pressure. The residue was dissolved in dicholomethane (6 mL), p-chloranil (4 mg, 0.0163 mmol) was added, the system was stirred for 1 hour at room temperature, and the solvent was then distilled out under reduced pressure. Trifluoroacetic acid (4 mL) was added to the residue, the system was stirred for 1 hour at room temperature, and the solvent was then distilled out under reduced pressure. The residue was refined using HPLC (eluent, from 32% acetonitrile/0.1% trifluoroacetic acid/water (0 minutes) to 64% acetonitrile/0.1% TFA/water (30 minutes); flow rate=5.0 mL/min) to obtain Suc-LLVY-SiR600 (2.8 mg, 2.68 μmol, yield: 26%).

HRMS (ESI⁺): m/z Found 931.4832. calculated 931.4790 for [M]⁺ (−4.2 mmu)

A single peak was observed at 12.8 minutes using a HPLC chromatogram after refinement (from 40% acetonitrile/0.1% trifluoroacetic acid/water to 80% acetonitrile/0.1% TFA/water; flow rate=1.0 mL/min), linear gradient, Abs. 500 nm).

### (2) Synthesis of Suc-TPLL-SiR600

### Example 2

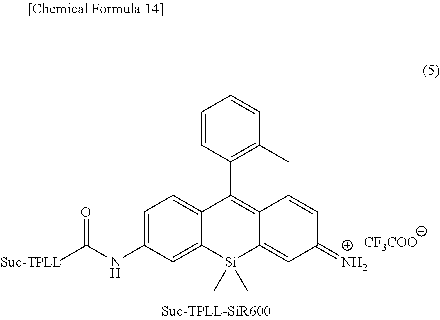

9-o-toluyl-9H-Si-xanthene-3,6-diamin (3.71 mg, 10.8 μmol) was dissolved in DMF (0.5 mL), side-chain-protected peptide 4 (7.9 mg, 12.1 μmol), HATU (9.04 mg, 23.8 μmol), and DIPEA (8.3 μL, 47.5 μmol) were added, and the system was stirred for 23 hours at room temperature. Water was added to the reaction mixture, and the resulting mixture was extracted using dichloromethane and washed using a saline solution. The organic layer was dried using sodium sulfate, and the solvent was then distilled out under reduced pressure. The residue was dissolved in dicholomethane (6 mL), p-chloranil (5 mg, 0.02 mmol) was added, the system was stirred for 12 hours at room temperature, and the solvent was then distilled out under reduced pressure. Trifluoroacetic acid (3 mL) was added to the residue, the system was stirred for 2 hours at room temperature, and the solvent was then distilled out under reduced pressure. The residue was refined using HPLC (eluent, from 40% acetonitrile/0.1% trifluoroacetic acid/water (0 minutes) to 80% acetonitrile/0.1% TFA/water (20 minutes); flow rate=5.0 mL/min) to obtain Suc-TPLL-SiR600 (2.8 mg, 2.86 μmol, yield: 26%).

HRMS (ESI⁺): m/z Found 867.4480. calculated 867.4477 for [M]⁺ (+0.3 mmu)

A single peak was observed at 10.6 minutes using a HPLC chromatogram after refinement (from 40% acetonitrile/0.1% trifluoroacetic acid/water to 80% acetonitrile/0.1% TFA/water; flow rate=1.0 mL/min), linear gradient, Abs. 500 nm).

### (3) Synthesis of Boc-LM-SiR600

### Example 3

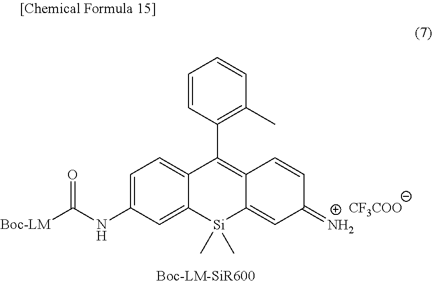

9-o-toluyl-9H-Si-xanthene-3,6-diamin (3.9 mg, 11.3 μmol) was dissolved in DMF (1.5 mL), side-chain-protected peptide 6 (4.5 mg, 12.5 μmol), HATU (9.5 mg, 24.9 μmol), and DIPEA (8.3 μL, 49.7 μmol) were added, and the system was stirred for 24 hours at room temperature. Water was added to the reaction mixture, and the resulting mixture was extracted using dichloromethane and washed using a saline solution. The organic layer was dried using sodium sulfate, and the solvent was then distilled out under reduced pressure. The residue was dissolved in dicholomethane (10 mL), p-chloranil (4 mg, 0.0163 mmol) was added, the system was stirred for 2 hours at room temperature, and the solvent was then distilled out under reduced pressure. The residue was refined using HPLC (eluent, from 40% acetonitrile/0.1% trifluoroacetic acid/water (0 minutes) to 80% acetonitrile/0.1% TFA/water (20 minutes); flow rate=5.0 mL/min) to obtain Boc-LM-SiR600 (0.6 mg, 0.75 μmol, yield: 7%).

HRMS (ESI⁺): m/z Found 687.3440. calculated 687.3400 for [M]⁺ (−4.0 mmu)

A single peak was observed at 17.0 minutes using a HPLC chromatogram after refinement (from 40% acetonitrile/0.1% trifluoroacetic acid/water to 80% acetonitrile/0.1% TFA/water; flow rate=1.0 mL/min), linear gradient, Abs. 500 nm).

### Example 4

### Measurement of the Optical Characteristics of Suc-LLVY-SiR600 and Boc-LM-SiR600

The optical characteristics of Suc-LLVY-SiR600 and Boc-LM-SiR600 were measured in a 0.1 sodium phosphate buffer with pH 3 containing 1% DMSO. The optical characteristics of 2MeSiR600, which is an enzyme reaction product, are also shown in TABLE 1.

Suc-LLVY-SiR600 and Boc-LM-SiR600 did not absorb light near maximum absorption (593 nm) of 2MeSiR600 generated by contact with calpain, and it was confirmed that measurement of calpain activity, which uses excitation light near 593 nm, could be carried out without influence from Suc-LLVY-SiR600 and Boc-LM-SiR600.

TABLE 1

| | Absₘₐₓ | Emₘₐₓ | | |
|---|---|---|---|---|
| | [nm] | [nm] | Φₙ | ε (M⁻¹cm⁻¹) |
| Suc-LLVY-SiR600 | 497 | 590 | 0.03 | 25,000 |
| Boc-LM-SiR600 | 495 | 589 | 0.12 | 26,000 |
| 2Me SiR600 | 593 | 613 | 0.38 | 91,000 |

### Example 5

### Evaluation of Suc-LLVY-SiR600 as a Fluorescent Probe

The Suc-LLVY-SiR600 obtained in Example 1 was evaluated as a calpain fluorescent probe.

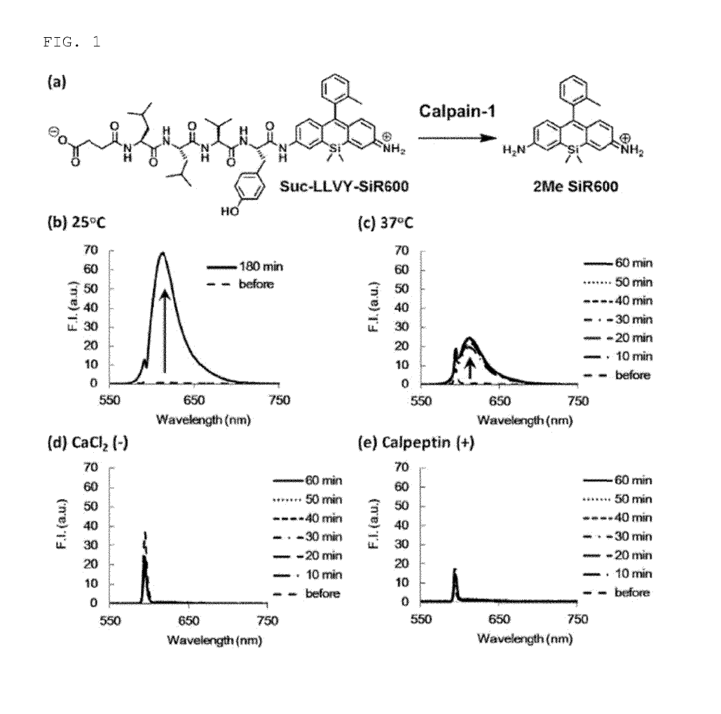

FIG. 1(a) shows the reaction scheme between calpain-1 and Suc-LLVY-SiR600.

FIG. 1(b) shows the fluorescent spectrum before addition of calpain-1 to the Suc-LLVY-SiR600 solution (2 μM) and the fluorescence spectrum 180 minutes later from addition of 5 μg calpain-1.

FIGS. 1(c) to 1(e) show the fluorescence spectrum 10 to 60 minutes later from addition of 5 μg calpain-1 to the Suc-LLVY-SiR600 solution (2 μM).

In FIGS. 1(b) to 1(e), the reaction was carried out in a 0.75-mL of 20-mM HEPES buffer solution (pH 7.4) containing 100-μM DTT, 10% glycerol, 0.1% CHAPS, 100-mM NaCl, 1-mM EDTA, and 1.5-mM CaCl₂, and containing 1% DMSO as a cosolvent.

In FIG. 1(d), the reaction was carried out in the absence of 1.5-mM CaCl₂, and in FIG. 1(e), the reaction was carried out in the presence of 1-μM calpeptin.

The reaction temperature was 25° C. in FIGS. 1(b), 1(d), and 1(e), and 37° C. in FIG. 1(c).

Although the maximum absorption wavelength of Suc-LLVY-SiR600 was near 500 nm as shown in TABLE 1, the measurement was taken using excitation light at 593 nm before and after the reaction with calpain since 2Me SiR600 generated by the reaction of Suc-LLVY-SiR600 and calpain has a maximum absorbance at 593 nm.

As a result, fluorescence was mostly unobserved before the reaction, but high-intensity fluorescence was observed after the reaction, as shown in FIG. 1(b). It was therefore shown that Suc-LLVY-SiR600 can be advantageously used as a fluorescent probe in relation to calpain. Also, as shown in FIG. 1(c), autolysis of calpain-1 was more rapid when the reaction solution was 37° C. than it was at 25° C., and the increase in fluorescence intensity therefore stopped more quickly.

Calpain shows enzymatic activity when bonded to Ca²⁺. It was confirmed that an increase in fluorescence does not occur in a solution that does not contain Ca²⁺ (FIG. 1(d)). Furthermore, FIG. 1(e) shows that an increase in fluorescence does not occur when calpeptin is added, calpeptin being a calpain selective inhibitor.

### Example 6

### Evaluation of Boc-LM-SiR600 as a Fluorescent Probe

The Boc-LM-SiR600 obtained in Example 3 was evaluated as a calpain fluorescent probe.

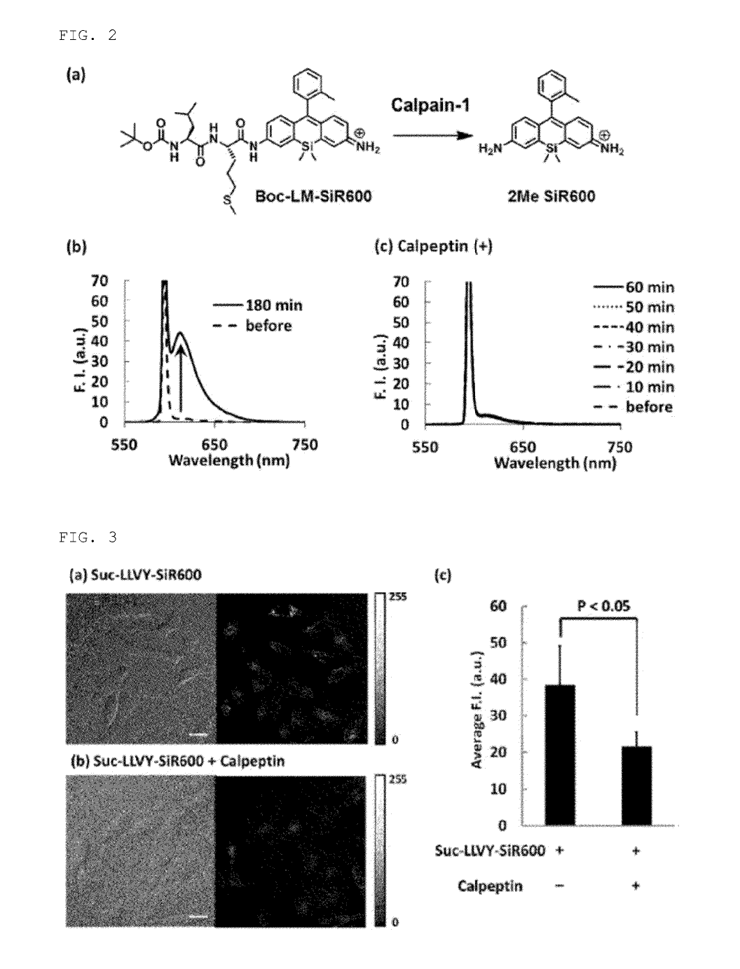

FIG. 2(a) shows the reaction scheme between calpain-1 and Boc-LM-SiR600.

FIG. 2(b) shows the fluorescence spectrum before addition of calpain-1 to the Boc-LM-SiR600 solution (2 μM) and the fluorescence spectrum 180 minutes later from addition of 5 μg calpain-1. FIG. 2(c) shows the fluorescence spectrum 10 to 60 minutes later from addition of 5 μg calpain-1 to the Boc-LM-SiR600 solution (2 μM).

In FIGS. 2(b) and 2(c), the reaction was carried out in a 0.75-mL of 20-mM HEPES buffer solution (pH 7.4) containing 100-μM DTT, 10% glycerol, 0.1% CHAPS, 100-mM NaCl, 1-mM EDTA, and 1.5-mM CaCl₂, and containing 1% DMSO as a cosolvent. In FIG. 2(c), the reaction was carried out in the presence of 1-μM calpeptin, and in FIG. 2(b), the reaction was carried out in the absence of calpeptin.

The reaction temperature was 25° C. in FIGS. 2(b) and 2(c), and the excitation wavelength was 593 nm.

As shown in FIG. 2(b), Boc-LM-SiR600 exhibited an increase in fluorescence by addition of calpain-1. Also, an increase in fluorescence did not occur when calpeptin was added, calpeptin being a calpain selective inhibitor (FIG. 2(c)).

### Example 7

### Imaging Using Suc-LLVY-SiR600 in Living Cells

### (1) Application to Imaging of Calpain Activity in HeLa Cells.

Calpain activity in HeLa cells was visualized using Suc-LLVY-SiR600.

Hela cells were incubated for 10 minutes at 37° C. using (a) Hank's balanced salt solution (HBSS) containing DMSO as a control, and (b) HBSS containing 20-11M calpeptin, and further incubation for 20 minutes was carried out in 2-μM Suc-LLVY-SiR600. A fluorescence image and a differential interference image were then taken using a confocal microscope. The results are shown in FIGS. 3(a) and 3(b). The scale bar in the drawings is 20 μm.

As shown in FIG. 3(a), calpain activity in HeLa cells can be monitored by adding Suc-LLVY-SiR600 to the extracellular fluid. Also, as shown in FIG. 3(b), fluorescence intensity in the cells was reduced by addition of calpeptin, which is a calpain selective inhibitor.

FIG. 3(c) shows the average fluorescence intensity in the Hela cells in three experiments in the presence or absence of calpeptin. Statistical analysis was carried out using Student's t-test (n=18). The error bar shows the standard deviation.

### (2) Application to Imaging of Calpain Activity in A549 Cells

Calpain activity in A549 cells was visualized using Suc-LLVY-SiR600.

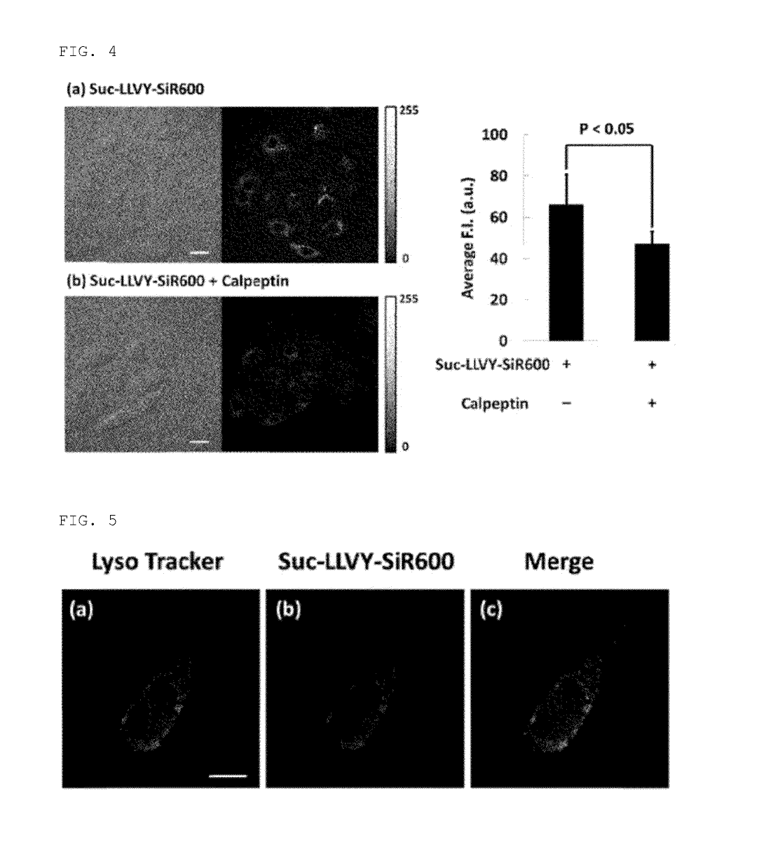

A549 cells were incubated for 10 minutes at 37° C. using (a) Hank's balanced salt solution (HBSS) containing DMSO as a control, and (b) HBSS containing 20-μM calpeptin, and further incubation for 20 minutes was carried out in 2-μM Suc-LLVY-SiR600. A fluorescence image and a differential interference image were then taken using a confocal microscope. The results are shown in FIGS. 4(a) and 4(b). The scale bar in the drawings is 20 μm.

FIG. 4(c) shows the average fluorescence intensity in the A549 cells in three experiments in the presence or absence of calpeptin. Statistical analysis was carried out using Student's t-test (n=18). The error bar shows the standard deviation.

As shown in FIG. 4(a), calpain activity in cells was successfully visualized using Suc-LLVY-SiR600 A549 cells as well.

### (3) Intracellular Localization of Dye

Costaining was carried out using the lysosomal localization dye LysoTracker and Suc-LLVY-SiR600.

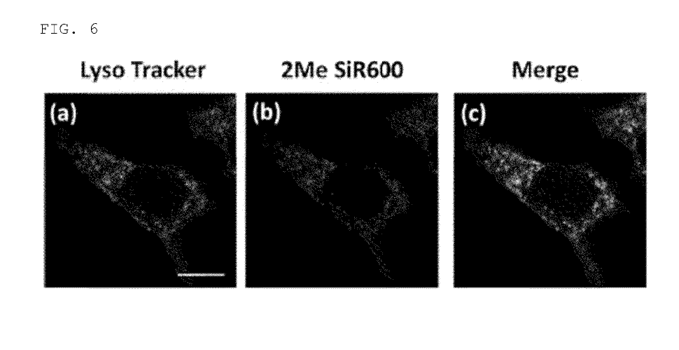

HeLa cells were incubated for 30 minutes in Suc-LLVY-SiR600 (2 μM), and the cells were allowed to uptake LysoTracker Green DND 26 (50 nM). A fluorescence image was then taken using a confocal microscope. The results are shown in FIG. 5.

In FIG. 5(a), the excitation wavelength/detection wavelength is 504 nm/514-534 nm, and in FIG. 5(b), the excitation wavelength/detection wavelength is 593 nm/603-623 nm. The scale bar in the drawings is 10 μm.

Next, costaining was carried out using LysoTracker and 2Me SiR600.

HeLa cells were incubated using 2Me SiR600 (50 μM) and LysoTracker Green DND 26 (50 nM). A fluorescence image was then taken using a confocal microscope. The results are shown in FIG. 6. In FIG. 6(a), the excitation wavelength/detection wavelength is 504 nm/520-550 nm, and in FIG. 6(b), the excitation wavelength/detection wavelength is 593 nm/608-638 nm. FIG. 6(c) shows a merged image of the preceding two images. The scale bar in the drawings is 10 μm.

Thus, it was demonstrated that Suc-LLVY-SiR600 shows intracellular localization in similar fashion to the lysosomal localization dye LysoTracker, and 2Me SiR600, which is an enzyme reaction byproduct of a probe, accumulates in lysosomes in cells. Therefore, Suc-LLVY-SiR600 can be effectively used for visualization of calpain activity in living cells.

1. A compound represented by general formula (I) below or a salt thereof: 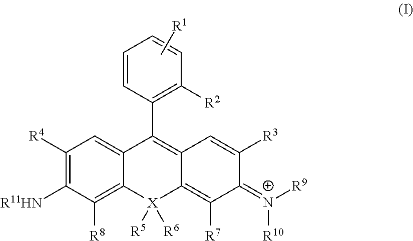
wherein
R¹ is a hydrogen atom or represents 1 to 4 monovalent substituents present on a benzene ring, which are the same or different;
R² is a monovalent substituent;
R³ and R⁴ are, independently, a hydrogen atom or C₁₋₆ alkyl group;
R⁵ and R⁶ are, independently, a C₁₋₆ alkyl group or aryl group;
R⁷ and R⁸ are, independently, a hydrogen atom or C₁₋₆ alkyl group;
R⁹ and R¹⁰ are, independently, a hydrogen atom or C₁₋₆ alkyl group;
R⁹ or R¹⁰ optionally forms, together with R³ or R⁷, a 5-7-member heterocyclyl or heteroaryl containing a nitrogen atom to which R⁹ or R¹⁰ binds, and optionally contains 1-3 additional heteroatoms selected from the group consisting of O, N, and S as ring-constituting members; the heterocyclyl or heteroaryl optionally being substituted by a C₁₋₆ alkyl, C₂₋₆ alkenyl, C₂₋₆ alkynyl, C₆₋₁₀ aralkyl, or C₆₋₁₀ aralkenyl group;
R¹¹ is a monovalent substituent which is to be cleaved by contact with calpain; and
X is a silicon, germanium, or tin atom.

2. The compound or salt thereof according to claim 1, wherein R¹¹ is a monovalent substituent containing an oligopeptide residue.

3. The compound or salt thereof according to claim 2, wherein the monovalent substituent containing an oligopeptide residue is represented by formulas (1), (2), or (3) below:

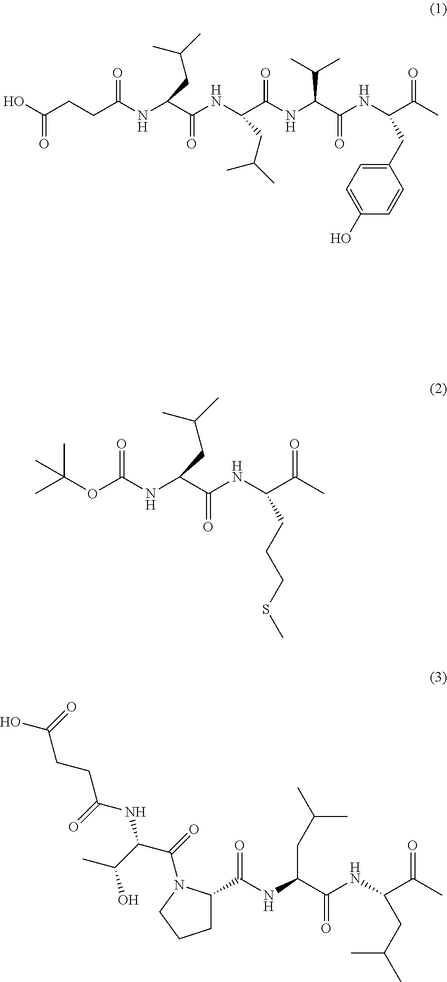

4. The compound or salt thereof according to claim 1, wherein X is a silicon or germanium atom.

5. A compound represented by formula (4) below, or salt thereof:

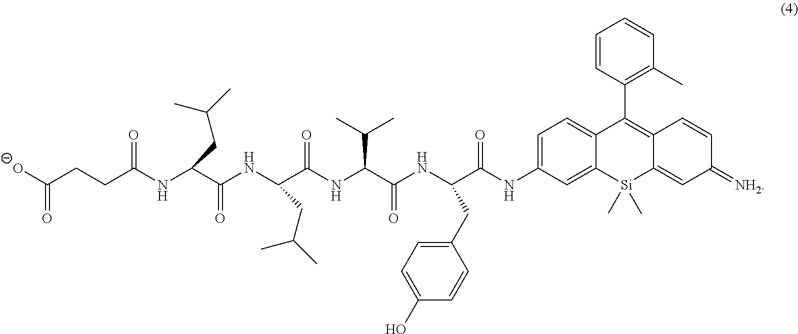

6. A compound represented by formula (5) below, or salt thereof:

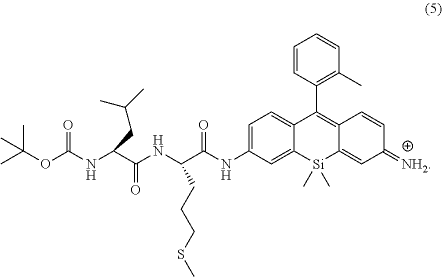

7. A compound represented by formula (6) below, or salt thereof:

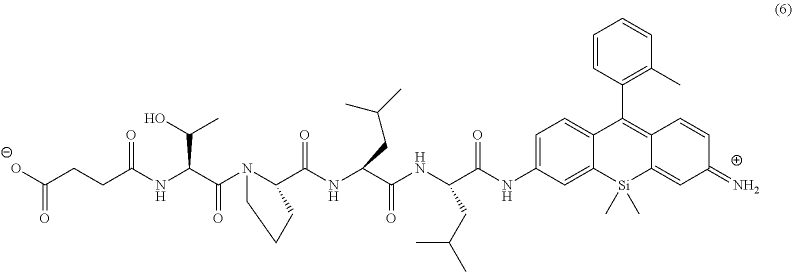 [SMILES: Cc1ccccc1C1=C2C=CC(=N)C=C2[Si](C)(C)c2cc(NC(=O)[C@H](CC(C)C)NC(=O)[C@H](CC(C)C)NC(=O)[C@@H]3CCCN3C(=O)[C@@H](NC(=O)CCC(=O)[O-])C(C)O)ccc21]

8. A fluorescent probe containing the compound or salt thereof according to claim 1.

9. A method for measuring calpain, comprising:
(a) bringing the compound or salt thereof according to claim 1 and calpain into contact with each other, and
(b) measuring the fluorescence intensity of the compound generated in step (a) after contact with calpain.

10. The compound or salt thereof according to claim 2, wherein X is a silicon or germanium atom.

11. The compound or salt thereof according to claim 3, wherein X is a silicon or germanium atom.

12. A fluorescent probe containing the compound or salt thereof according to claim 2.

13. A fluorescent probe containing the compound or salt thereof according to claim 3.

14. A fluorescent probe containing the compound or salt thereof according to claim 4.

15. A fluorescent probe containing the compound or salt thereof according to claim 5.

16. A fluorescent probe containing the compound or salt thereof according to claim 6.

17. A fluorescent probe containing the compound or salt thereof according to claim 7.

18. A method for measuring calpain, comprising:
(a) bringing the compound or salt thereof according to claim 5 and calpain into contact with each other, and
(b) measuring the fluorescence intensity of the compound generated in step (a) after contact with calpain.

19. A method for measuring calpain, comprising:
(a) bringing the compound or salt thereof according to claim 6 and calpain into contact with each other, and
(b) measuring the fluorescence intensity of the compound generated in step (a) after contact with calpain.

20. A method for measuring calpain, comprising:
(a) bringing the compound or salt thereof according to claim 7 and calpain into contact with each other, and
(b) measuring the fluorescence intensity of the compound generated in step (a) after contact with calpain.
